(19) United States
(12) Patent Application Publication
(10) Pub. No.: US 2006/0088581 A1
(43) Pub. Date: Apr. 27, 2006

(54) Device for delivery of therapeutic agent

(76) Inventors: Thomas Blaszczykiewicz, Orchard Park, NY (US); Tamer Elsamahy, Amherst, NY (US)

Correspondence Address: HODGSON RUSS LLP, ONE M & T PLAZA, SUITE 2000, BUFFALO, NY, 14203-2391, US

(21) Appl. No.: 11/232,638

(22) Filed: Sep. 22, 2005

Related U.S. Application Data
(60) Provisional application No. 60/612,306, filed on Sep. 22, 2004.
(60) Provisional application No. 60/656,041, filed on Feb. 24, 2005.

(51) Int. Cl. A61K 9/70; A61F 5/00

(52) U.S. Cl. 424/449; 602/1

(57) ABSTRACT

The invention provides a system for administering to or through the skin of the individual selected medicaments. The system includes an orthosis and a pad. The pad includes at least one delivery station suitable for containing the medicament. The pad may be positioned in a variety of configurations on the band dependant upon the body part to which the orthosis is applied. Additionally, the pad may comprise a plurality of delivery stations to enhance the spatial and/or temporal parameters for medicament delivery. Also provided is a method for delivering a medicament to or through a selected location of the skin of an individual comprising the steps of attaching to the individual the delivery system of the invention such that the medicament is delivered to or through the skin of the individual.

This application claims priority to U.S. provisional application No. 60/656,041, filed on Feb. 24, 2005, and U.S. provisional application No. 60/612,306, filed Sep. 22, 2004, the disclosures of which are incorporated herein by reference.

### FIELD OF THE INVENTION

The invention relates generally to the field of devices for compressing an area of the body and for delivery of medicaments to or through the skin.

### SUMMARY OF THE INVENTION

In the present invention is provided a system for compressing an area of the body of an individual and for administering to or through the skin of the individual one or more selected medicaments for treating a variety of ailments. The system comprises an orthosis and a pad which pad comprises at least one delivery station suitable for containing the medicament. In particular embodiments, the orthosis can be suitable for compression of any area of the body, such as joints, limbs, head neck or trunk. The orthosis may accommodate a variety of pad configurations dependant upon the body part to which the orthosis is applied. Additionally, the pad may comprise a plurality of delivery stations which can be provided in various configurations to enhance the spatial and/or temporal parameters for medicament delivery.

Also provided is a method for delivering a medicament to or through a selected location of the skin of an individual comprising the steps of attaching to the individual the delivery system of the invention such that the medicament is delivered to or through the skin of the individual.

### BRIEF DESCRIPTION OF THE DRAWINGS

The invention is illustrated in the drawings in which like reference characters designate the same or similar parts throughout the figures of which:

FIG. 1 is a top plan view of one embodiment of the device present invention.

FIG. 2 is a perspective view of one embodiment of the device of the present invention;

### DETAILED DESCRIPTION

In the present invention is provided a system for delivering a medicament to a selected location on the skin of an individual. “Medicament” as used herein means a substance used in therapy. As such, the medicament used in the present invention can be any substance suitable for delivery to or through the skin of an individual to provide therapy for a variety of ailments. For example, the medicaments can include, but are not limited to, analgesics, such as acetaminophen or aspirin, anti-nausea agents, such as Dramamine, antimicrobial agents, vitamins, and herbal formulations, such as white willow, glucosamine, chondroitin, creatine, insulin or the like. Further, a medicament may also be considered to comprise reagents used in formulating the composition that is delivered to the skin.

In another embodiment, the invention provides a method for delivering a medicament to or through a selected location of the skin of an individual. The method comprises attaching to the individual a system comprising an orthosis and a pad, wherein the pad comprises at least one medicament delivery station which contains a medicament, such that the medicament is delivered to or through the skin of the individual.

In general, the system comprises an orthosis and a pad. The pad comprises at least one medicament delivery station, also referred to herein as a “delivery station.” The orthosis functions to compress the area of the body to which it is applied, thereby bringing the delivery station(s) into intimate contact with the skin of the individual. In particular embodiments described more fully below, one or more delivery stations may be provided as part of the pad or affixed to a substrate on the pad. In this regard, the pad may provide support for a substrate into which the delivery stations are set, or the delivery stations may be formed into the pad itself. In one embodiment, the entire surface of the pad may constitute a single delivery station.

The pad may be formed of any suitable material, such as rubber, nylons, synthetic or natural fibers, polymers, etc. In a preferred embodiment, the pad is formed from polyurethane. However, as will be clear to those skilled in the art, the pad, regardless of the substance from which is constructed, may be prepared by conventional methods.

The pad is preferably selectively configurable on the surface of the orthosis that contacts the individual. By configurable it is meant that the pad can be positioned at multiple locations on the orthosis such that the pad and concomitant delivery station(s) can be positioned on the orthosis such that the medicament is delivered to an optimal location on the individual.

The delivery station(s) may be flat, recessed, such as a well, or raised, such as a dimple. The delivery stations may further comprise a removable covering such that each delivery station can be selectively activated to deliver its contents by removing the covering and permitting contact between the medicament and the skin. The delivery stations may also independently or additionally comprise a permeable membrane for regulating the flow rate of the medicament to the skin. A rate-controlling membrane, if present, will be included in the system on the skin side of one or more of the medicament stations. The materials used to form such a membrane are selected to limit the flux of one or more components contained in the medicament formulation. Representative materials useful for forming rate-controlling membranes include polyolefins such as polyethylene and polypropylene, polyamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, and the like.

The orthosis can be any conventional orthosis so long as it compresses the area of the body to which it is applied and has been adapted to receive the pad comprising the delivery station(s). Examples of suitable orthosis include but are not limited to adjustable back braces, slip on or adjustable knee braces, slip on or adjustable ankle braces, slip on or adjustable elbow braces, and the like. In a preferred embodiment, the orthosis is a conventional band-type brace suitable for compressing a limb or the trunk of the individual.

In respect of the medicament, it is preferable for the medicament to be provided with a pharmaceutically acceptable topical carrier. In this regard, the medicament/carrier formulation may be in any form suitable for application to the body surface, and may comprise, for example, a cream, lotion, solution, gel, a hydrogel, an ointment, paste, or the like, and/or may be prepared so as to contain liposomes, micelles, and/or microspheres. Such a formulations may be aqueous, i.e., contain water, or may be nonaqueous and optionally used in combination with an occlusive overlayer so that moisture evaporating from the body surface is maintained within the formulation upon application to the body surface and thereafter.

Ointments are semisolid preparations that are typically based on petrolatum or other petroleum derivatives. The specific ointment base to be used is one that will provide for optimum medicament delivery, and, preferably, will provide for other desired characteristics as well, e.g., emolliency or the like. As with other carriers or vehicles, an ointment base should be inert, stable, nonirritating and nonsensitizing. As is known to those skilled in the art, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin, and hydrophilic petrolatum. Emulsion ointment bases are either water-in-oil (W/O) emulsions or oil-in-water (O/W) emulsions, and include, for example, cetyl alcohol, glyceryl monostearate, lanolin, and stearic acid. Water-soluble ointments include those prepared from polyethylene glycols of varying molecular weight. See Remington: The Science and Practice of Pharmacy for further information.

Creams, as also well known in the art, are viscous liquids or semisolid emulsions, either oil-in-water or water-in-oil. Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant.

Gels are semisolid, suspension-type systems. Single-phase gels contain organic macromolecules distributed substantially uniformly throughout the carrier liquid, which is typically aqueous, but also, preferably, contains an alcohol and, optionally, an oil. The gelling agents may be crosslinked acrylic acid polymers such as the “carbomer” family of polymers, e.g., carboxypolyalkylenes that may be obtained commercially under the Carbopol® trademark. Also included for use with the invention are hydrophilic polymers such as polyethylene oxides, polyoxyethylene-polyoxypropylene copolymers, and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by titration, mechanical mixing, or stirring, or combinations thereof.

In one embodiment, the medicament is provided in a hydrogel. A hydrogel is generally a blend of at least two polymers one of which is a crosslinked polymer derived from one or more olefinically unsaturated polymerizable carboxylic monomers and optionally one or more comonomers. The other polymer is a high molecular weight substantially linear polymer derived from one or more olefinically unsaturated polymerizable carboxylic acid monomers. The hydrogel also contains a neutralizing agent; desirably a noncovalent crosslinking agent; and a cure rate modifier; and may be located on a backing with a release compound or liner covering the hydrogel. The hydrogel is typically located on a substrate, such as the pad of the present invention. Additionally, the hydrogel can comprise any natural or synthetic polymer that is highly swollen by water. Polymers forming the hydrogel may be gel-like particles that have the capacity to increase their volume by swelling in water by a factor from about 10 to about 1000 or 10,000.

Lotions are preparations to be applied to the skin surface without friction, and are typically liquid or semiliquid preparations in which solid particles, including the medicament, are present in a water or alcohol base. Lotions are usually suspensions of solids and may comprise a liquid oily emulsion of the oil-in-water type. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like.

Pastes are semisolid dosage forms in which the medicament is suspended in a suitable base. Depending on the nature of the base, pastes are divided between fatty pastes or those made from a single-phase aqueous gels. The base in a fatty paste is generally petrolatum, hydrophilic petrolatum, or the like. The pastes made from single-phase aqueous gels generally incorporate carboxymethylcellulose or the like as a base.

The medicament may also be prepared with liposomes, micelles, or microspheres. Liposomes are microscopic vesicles having a lipid wall comprising a lipid bilayer, and can be used as drug delivery systems with the present invention. Generally, liposome formulations are preferred for poorly soluble or insoluble pharmaceutical agents. Liposomal preparations for use in the instant invention include cationic (positively charged), anionic (negatively charged), and neutral preparations. Cationic liposomes are readily available. For example, N[1-2,3-dioleyloxy)propyl]-N,N,N-triethylammonium (DOTMA) liposomes are available under the tradename Lipofectin® (GIBCO BRL, Grand Island, N.Y.). Similarly, anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), and dioleoylphoshatidyl ethanolamine (DOPE), among others. These materials can also be mixed with DOTMA in appropriate ratios. Methods for making liposomes using these materials are well known in the art.

Micelles are known in the art as comprised of surfactant molecules arranged so that their polar headgroups form an outer spherical shell, while their hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethylammonium chloride, dodecylammonium chloride, polyoxyl 8 dodecyl ether, polyoxyl 12 dodecyl ether, nonoxynol 10, and nonoxynol 30. Micelle formulations can be used in conjunction with the present invention either by incorporation into the reservoir of a topical or transdermal delivery system, or into a formulation to be applied to the body surface.

Microspheres, similarly, may be incorporated into the present formulations and drug delivery systems. Like liposomes and micelles, microspheres essentially encapsulate a medicament containing formulation. Microspheres are generally, although not necessarily, formed from synthetic or naturally occurring biocompatible polymers, but may also be comprised of charged lipids such as phospholipids. Preparation of microspheres is well known in the art and described in the pertinent texts and literature.

Various additives, known to those skilled in the art, may be included in the topical formulations for use with the invention. For example, solvents, including relatively small amounts of alcohol, may be used to solubilize certain formulation components. Although the medicaments descried herein may penetrate into the skin, it may be desirable to include an added permeation enhancer in the formulation if enhanced penetration is desired. Examples of suitable enhancers include, but are not limited to, ethers such as diethylene glycol monoethyl ether (available commercially as Transcutol®) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80), and lecithin (U.S. Pat. No. 4,783,450); alcohols such as ethanol, propanol, octanol, benzyl alcohol, and the like; polyethylene glycol and esters thereof such as polyethylene glycol monolaurate (PEGML; see, e.g., U.S. Pat. No. 4,568,343); amides and other nitrogenous compounds such as urea, dimethylacetamide (DMA), dimethylformamide (DMF), 2-pyrrolidone, 1-methyl-2-pyrrolidone, ethanolamine, diethanolamine, and triethanolamine; terpenes; alkanones; and organic acids, particularly citric acid and succinic acid. Azone® and sulfoxides such as DMSO and C₁₀MSO may also be used, but are less preferred. Additional permeation enhancers will be known to those of ordinary skill in the art of topical drug delivery, and/or are described in the pertinent texts and literature. See, e.g., Percutaneous Penetration Enhancers, eds. Smith et al. (CRC Press, 1995).

Formulations for use with the present invention may also include conventional additives such as opacifiers, antioxidants, fragrance, colorant, gelling agents, thickening agents, stabilizers, surfactants, and the like. Other agents may also be added, such as antimicrobial agents, to prevent spoilage upon storage, i.e., to inhibit growth of microbes such as yeasts and molds. Suitable antimicrobial agents include the methyl and propyl esters of p-hydroxybenzoic acid (i.e., methyl and propyl paraben), sodium benzoate, sorbic acid, imidurea, and combinations thereof.

The formulations may also contain irritation-mitigating additives to minimize or eliminate the possibility of skin irritation or skin damage resulting from the medicament or other components of the composition. Suitable irritation-mitigating additives include, for example: α-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. The irritant-mitigating additive, if present, may be incorporated into the present formulations at a concentration effective to mitigate irritation or skin damage, typically representing not more than about 20 wt. %, more typically not more than about 5 wt. %, of the composition.

In one embodiment, the pad, the delivery station, or both, comprise a pharmaceutically acceptable adhesive material that serves to affix the system to the skin during drug delivery. Typically, the adhesive material is a pressure-sensitive adhesive that is suitable for long-term skin contact, and that should be physically and chemically compatible with the medicament and any carriers or other additives that are present. Examples of suitable adhesive materials include, but are not limited to, the following: polyethylenes; polysiloxanes; polyisobutylenes; polyacrylates; polyacrylamides; polyurethanes; plasticized ethylene-vinyl acetate copolymers; and tacky rubbers such as polyisobutene, polybutadiene, polystyrene-isoprene copolymers, polystyrene-butadiene copolymers, and neoprene (polychloroprene).

The amount of medicament administered is an amount required to achieve an effective therapeutic result. Such an amount depends on many factors, such as the minimum necessary dosage of the medicament for the particular indication being treated; the solubility and permeability of the carrier and adhesive layer; and the period of time for which the device will be affixed to the skin. The minimum amount of medicament is determined by the requirement that a sufficient quantity of drug must be present in the device to maintain the desired rate of release over the given period of application. The maximum amount for safety purposes is determined by the requirement that the quantity of drug present cannot exceed a rate of release that reaches toxic levels. Generally, the maximum concentration is determined by the amount of agent that can be received in the carrier without producing adverse histological effects such as irritation, an unacceptably high initial pulse of agent into the body, or adverse effects on the characteristics of the delivery device such as the loss of tackiness, viscosity, or deterioration of other properties.

Polyurethanes may be used to form the pad and/or a substrate affixed to the pad for the purpose of forming the delivery stations. The polyurethanes may be thermoplastic or elastomeric polyurethanes. They may be hydrophobic, hydrophilic or amphiphilic depending on the medicamnet or other additives to be incorporated used. Polyurethanes and their precursors are well known to the art.

The invention will be further described by the following drawings and description thereof which are meant to illustrate particular embodiments of the invention and are not meant to be limiting.

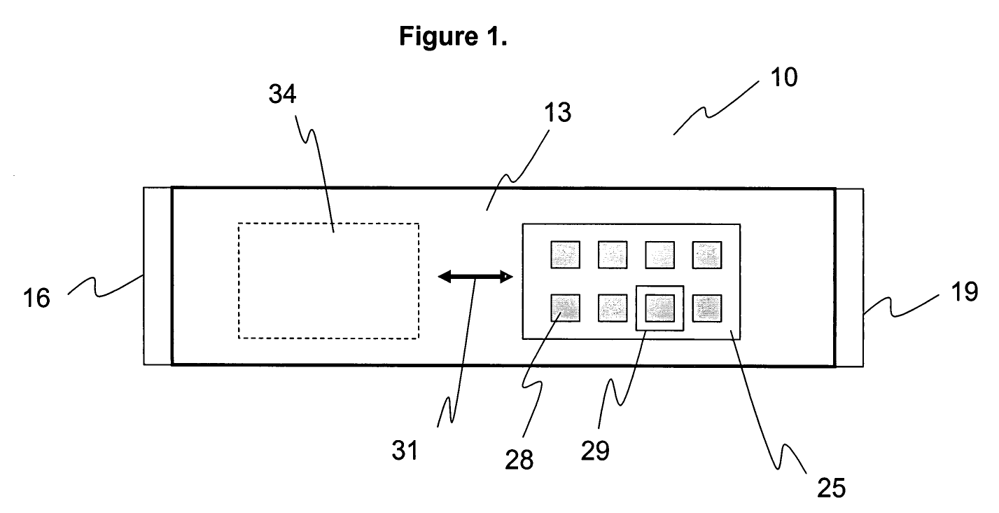

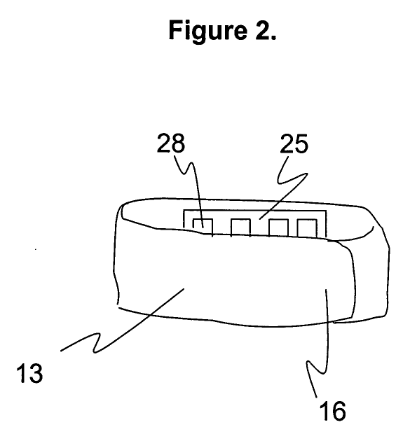

Referring to FIGS. 1-2, the device 10 of the present invention provides compression, optionally immobilization, and delivery of a medicament to or through the skin. In this embodiment, the device 10 includes a band 13 comprised of a fabric. It is preferred that the fabric is a synthetic stretchable fabric. One example of a suitable band fabric is a breathable neoprene substitute as disclosed in U.S. Pat. No. 6,861,379, which is incorporated herein by reference. The band 13 has a distal end 16 and a proximal end 19. Each of the distal end 16 and the proximal end 19 may comprise a means for fastening the band around a portion of an individual's body. For example, distal end 16 may include hooks on its outer surface and proximal end 19 may comprise loops on its inner surface such that the band 13 can be adjustably fastened around a selected body part by contacting the hooks and loops in a conventional manner. Alternatively, the distal end 16 and the proximal end 19 may include conventional complementary mating fasteners, such as buttons, laces, snaps and the like. In another embodiment, one of the distal end 16 or the proximal end 19 may be attached to a ring. For example, if the proximal end 19 is attached to a ring, the band 13 may be wrapped around the elbow, knee, wrist, ankle, back, etc. of a person and the distal end 16 can be threaded through the ring 22. The distal end 16 can then be folded back and attached to the band 13. The distal end 16 can be attached to the band by means of hook and loop fasteners, buttons, snaps, or other fasteners or the like as will be understood by those skilled in the art.

The pad 25 also comprises a plurality of individual delivery stations 28 containing individual doses of the active ingredients. Each delivery station 28 contains a dose of the medicament and can be covered by peel off strips 29. Accordingly, multiple doses may be stored on a single pad 25 and the peel off strips 29 can be removed to start the delivery of an additional dose. Alternatively, the peel off strips 29 may be replaced by permeable membranes of the type more fully described above. Either the peel off strips 29 or permeable membranes may be affixed to the pad 25 by adhesive. Further, the delivery stations may be refillable with a medicament of choice after their contents has been applied to the skin.

When the pad 25 comprises a single delivery station, the pad 25 can be recharged by means of a sprayer type system. In one embodiment, a supply of the medicament can be stored in a spray bottle and the delivery station can be periodically sprayed with a coating of the medicament to recharge the system.

The pad 25 may can be configured on the band 13. For example, the pad 25 and the band 13 may comprise a hook and loop fastening system so that the pad 25 can be positioned anywhere along the band 13 to enable the user to position the pad 25 in the precise area of pain. For example, the pad 25 can be moved in the direction of arrows 31 and may be repositioned as indicated by broken lines 34. Further, the pad 25 is not restricted to moving in the direction specified by the arrows 31 but can be placed anywhere on the band 13 as is convenient for the user.

In addition to the hook and loop fastening system, the pad 25 can be removably attached in any position on the band 13 by a pressure sensitive adhesive as will be evident to those of ordinary skill in the art based on this disclosure.

The orthopedic device 10 provides compression and optionally immobilization in addition to delivery of the medicament. The device 10 can also be configured to provide heat or to provide heat and the active ingredient in combination. Many elements such as heat, vibration, sweat, and mechanical pressure from the band 13 may combine with the pad 25 to stimulate absorption of the medicament into the skin.

In the case of transdermal drug delivery by means of the device 10, the system may also include a map of target areas of the body where the pad 25 should be positioned in order to maximize the effects.

While the invention has been described in connection with certain embodiments, it is not intended to limit the scope of the invention to the particular forms set forth, but, on the contrary, it is intended to cover such alternatives, modifications, and equivalents as may be included within the spirit and scope of the invention.

What is claimed is:

1. A system for delivering a medicament to a selected location on the skin of an individual, the system comprising:
an orthosis and a pad, wherein the pad is selectively configurable on the orthosis, and wherein the pad comprises at least one medicament delivery station, wherein the medicament delivery station contains a medicament suitable for delivery to or through the skin of the individual.

2. The system of claim 1, wherein the pad comprises a plurality of medicament delivery stations.

3. The system of claim 1, wherein the at least one medicament station includes a removable cover.

4. The system of claim 2, wherein the medicament stations are covered with a permeable membrane.

5. The system of claim 1, wherein medicament is an analgesic.

6. The system of claim 1, wherein the medicament is selected from the group consisting of acetaminophen, aspirin, ibuprofen, anti-nausea agents, antimicrobial agents, vitamins, herbal formulations, glucosamine, chondroitin, insulin and combinations thereof.

7. The system of claim 1, wherein the medicament is mixed with a composition selected from the group consisting of a pharmaceutically acceptable carrier, a permeation enhancer, a hydrogel, liposomes, micelles, microspheres, or combinations thereof.

8. The system of claim 7, wherein the pharmaceutically acceptable carrier is selected from the group consisting of creams, lotions, gels, hydrogels, ointments, pastes, and combinations thereof.

9. The system of claim 8, wherein the pharmaceutically acceptable carrier is a hydrogel.

10. The system of claim 1, wherein the pad is formed from a composition selected from the group consisting of polyurethane, rubber, nylons, synthetic fibers, natural fibers, and polymers, or combinations thereof.

11. The system of claim 1, wherein the orthosis is selected from the group consisting of a band, an ankle brace, an elbow brace, a wrist brace, a knee brace, a neck brace, and a back brace.

12. The system of claim 1, wherein the pad is selectively configured on the orthosis by adhering the pad to a selected location on the orthosis using an adhesive or a hook and loop fastening system.

13. The system of claim 11, wherein the band is adapted to be fastened to the individual using complementary mating fasteners selected from the group consisting of buttons, laces, snaps and a hook and loop system.

14. The system of claim 1, wherein the orthosis is formed from a synthetic fabric or a natural fabric.

15. The system of claim 14, wherein the synthetic fabric is selected from the group consisting of rubber, nylon, neoprene and a breathable neoprene substitute.

16. The system of claim 1, wherein the pad, the medicament delivery station, or both, comprises a pharmaceutically acceptable adhesive material for adhering the pad to the skin.

17. A method for administering a medicament to an individual comprising the steps of selecting a location on the individual in need of therapy and attaching to the skin at the selected location a system comprising an orthosis and a pad, wherein the pad is selectively configurable on the orthosis, wherein the pad comprises at least one medicament delivery station which contains a medicament, such that the medicament is delivered to or through the skin of the individual at the selected location.

18. The method of claim 17, wherein the pad comprises a plurality of medicament delivery stations and wherein each delivery station includes a removable cover.

19. The method of claim 17, wherein the medicament is an analgesic.

20. A system for delivering a medicament to a selected location on the skin of an individual, the system comprising:
a brace, the brace being formed from a stretchable material, wherein the brace comprises a distal end and a proximal end, wherein the distal end comprises a first complementary fastener and the proximal end comprises a second complementary fastener, wherein the first complementary fastener and the second complementary fastener can be removably attached to each other such that the distal end of the brace and the proximal end of the brace are held together such that the brace is affixed to a limb or the trunk of the individual; and
a pad, wherein the pad is selectively configurable on the brace by means of an adhesive backing on the pad; wherein the pad comprises a plurality of medicament delivery stations, wherein the medicament delivery station contains a medicament suitable for delivery to or through the skin of the individual, and wherein each medicament station includes a removable cover.
